(19) United States
(12) Patent Application Publication
(10) Pub. No.: US 2006/0127421 A1
(43) Pub. Date: Jun. 15, 2006

(54) Treatment using D-threo methylphenidate

(75) Inventor: Vikram Khetani, Short Hills, NJ (US)

Correspondence Address: WOODCOCK WASHBURN LLP, ONE LIBERTY PLACE, 46TH FLOOR, 1650 MARKET STREET, PHILADELPHIA, PA, 19103, US

(73) Assignee: Celgene Corporation, Summit, NJ (US)

(21) Appl. No.: 11/298,093

(22) Filed: Dec. 8, 2005

Related U.S. Application Data
(60) Provisional application No. 60/634,562, filed on Dec. 9, 2004.

(51) Int. Cl. A61K 31/445; A61K 9/00

(52) U.S. Cl. 424/400; 514/317

(57) ABSTRACT

Methods for treating a disease responsive to the administration of methylphenidate and/or one or more isomers thereof, said method comprising identifying a patient suffering from a disease or disorder having a family history or diagnosis of tics or Tourette's Syndrome and administering to said patient a therapeutically effective amount of D-threo methylphenidate substantially free of the l-threo isomer and of erythro methylphenidates.

### CROSS REFERENCE TO RELATED APPLICATION

This application claims the benefit of U.S. Application No. 60/634,562, filed Dec. 9, 2004, the entirety of which is incorporated herein by reference.

### FIELD OF THE INVENTION

The present invention is directed, inter alia, to methods of treating a class of patients suffering from Tourette's Syndrome who also suffer from a disease responsive to the administration of D-threo methylphenidate or a salt thereof, such as, for example, D-threo methylphenidate hydrochloride. Also disclosed are methods of administering to such patients therapeutically effective amounts of D-threo methylphenidate substantially free of the l-threo isomer and free of erythro methylphenidates.

### BACKGROUND OF THE INVENTION

Tourette's Syndrome is a severe neurological disorder characterized by multiple facial and other body tics, usually beginning in childhood or adolescence and often accompanied by grunts and compulsive utterances. Its symptoms typically begin when children are in grade school with many outgrowing the condition after adolescence. Although there is no cure for Tourette's Syndrome, medication may alleviate some of the symptoms.

A tic is a sudden, rapid, repetitive movement (motor tic) or vocalization (vocal tic). Motor tics usually involve muscles in a single location of the face or upper body. There are two main types of tics. Simple tics involve one muscle group—for example, head shaking, eye blinking, sniffing, neck jerking, shoulder shrugging, and facial grimacing. Complex tics involve more than one muscle group—for example, self-hitting or self-biting, jumping and hopping, and twirling while walking.

Tics sometimes evolve over time from one simple type of tic to another or from a simple to a complex tic. In addition, some tics are slow and sustained rather than brief and rapid; some tics involve the lower body. Vocal tics also can be simple (coughing, throat clearing, barking) or complex (repeating words out of context, echoing what someone else has said, uttering obscenities).

Tics are thought to be inherited neurological disorders that affect the body's motor system. Tics also can be caused by head injury or drugs, such as certain types of stimulants. People with tic disorders describe an urge building up inside them before the tic appears. This buildup feeling is called a premonition. People with tics often feel relief after the tic is over. Although tics are involuntary, the urge sometimes can be suppressed for short periods with voluntary effort. A burst of tics often follows voluntary suppression, to relieve a buildup of the inner sensation.

Those suffering from Tourette's Syndrome or tics may also suffer from other ailments comprising Attention Deficit Disorder (ADD), Attention Deficit-Hyperactivity Disorder (ADHD), and/or one or more of a decrease in cognitive function, fatigue, and/or neurobehavioral slowing that is unrelated to the administration of analgesics, but may be related to an underlying cancer, the treatment of the cancer, or both. For example, some physicians have estimated that more than 50 percent of people with Tourette's Syndrome also have ADHD.

Central Nervous System (CNS) stimulants are often prescribed to treat ADHD. Currently, the drugs made from these stimulants may be contraindicated in classes of patients (a) suffering from Tourette's Syndrome or tics and/or (b) having a family history of Tourette's Syndrome or tics. Tourette's Syndrome has also been considered an adverse reaction to the administration of methylphenidate hydrochloride drugs. In the past, certain references may have suggested that those patients who have tics, Tourette's Syndrome, or a family history of Tourette's Syndrome or tics should not take drugs comprising methylphenidate. The present invention relates, inter alia, to the administration of methylphendiate to those patients who have tics, Tourette's Syndrome, or a family history of Tourette's Syndrome or tics.

Methylphenidate exists as four separate optical isomers as follows: 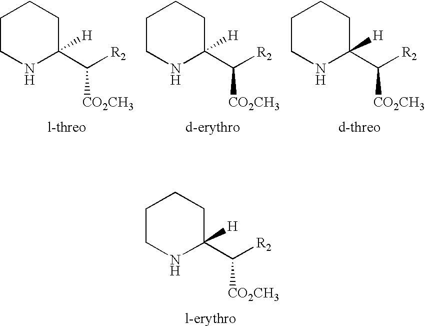
wherein R₂ is phenyl. Pharmaceutically acceptable salts are generally administered clinically.

The threo racemate (pair of enantiomers) of methylphenidate is a mild central nervous system stimulant with pharmacological activity qualitatively similar to that of amphetamines. Undesirable side effects associated with the use of the DL-threo racemate of methylphenidate comprise anorexia, weight loss, insomnia, dizziness, and dysphoria. Furthermore, the racemate, which is a Schedule II controlled substance, produces a euphoric effect when administered intravenously or through inhalation or ingestion, and thus carries a high potential for abuse.

Sustained release formulations of DL-threo methylphenidate have been developed, which provide for slow release of the drug over the course of the day. However, it has been observed that peak plasma concentrations of the drug are lower when sustained release formulations are used as compared to conventional dosage forms administered throughout the day. In some studies, sustained release formulations of DL-threo methylphenidate have been shown to have lower efficacy than conventional dosage forms.

Pulsed-release dosage forms, wherein a single dosage form contains two doses, one of which is released shortly after ingestion and the other of which is released following a delay of several hours, have recently been proposed as a method for administering a maximally effective dose regime. While pulsed dosage forms provide for efficient release of multiple doses of medication at predetermined intervals, such dosage forms can be complex and expensive to manufacture. However, it is desirable to administer to all patients the most effective and efficient dosage of mediation and, in the case of methylphenidate, it is now believed that this end is best achieved by administering the single, effective isomer, i.e. D-threo methylphenidate.

It has been discovered that the use of the D-threo isomer (2R:2′R) of methylphenidate, substantially free of the l-threo isomer and of erythro methylphenidates, produces a methylphenidate medication which retains high activity levels and simultaneously may possess reduced euphoric effect and reduced potential for abuse among patients. See U.S. Pat. No. 5,908,850, incorporated herein by reference in its entirety. Thus, D-threo methylphenidate (2R:2′R) may possess enhanced therapeutic activity with reduced side effects, and L-threo-methylphenidate may produce undesirable side effects, euphoria, and drug abuse potential in patients.

There remains a need for improved methods for treating patients suffering from Tourette's Syndrome in conjunction with another disorder that responds to D-threo methylphenidate. This invention is directed to these, as well as other, important ends.

### SUMMARY OF THE INVENTION

Without being limited by theory, the present invention relates, in part, to the hypothesis that D-threo methylphenidate, substantially free of the l-threo isomer and of erythro methylphenidates, may be safely administered to a class of patients suffering from Tourette's Syndrome and/or tics, along with ADD, ADHD, or other disorders responsive to the administration of D-threo methylphenidate or a salt thereof, such as, for example, D-threo methylphenidate hydrochloride. The other disorders may comprise one or more of a decrease in cognitive function, fatigue, and neurobehavioral slowing that is unrelated to the administration of analgesics, but may be related to an underlying cancer, the treatment of the cancer, or both. The present invention discloses methods for treating a class of patients rather than merely covering the treatment of certain indications. To that end, disclosed are methods for treating a disease or disorder responsive to the administration of D-threo methylphenidate or a salt thereof, such as, for example, D-threo methylphenidate hydrochloride, said method comprising the steps of identifying a patient suffering from such a disease or disorder and having a family history or diagnosis of tics or Tourette's Syndrome and administering to said patient a dosage form comprising a therapeutically effective amount of D-threo methylphenidate substantially free of the l-threo isomer and of erythro methylphenidates. Other embodiments are methods of treating a patient diagnosed with attention deficit disorder or attention deficit hyperactivity disorder and exhibiting tics or has a family history of Tourette's Syndrome comprising identifying a patient and administering to the patient a dosage form comprising a therapeutically effective amount of D-threo methylphenidate substantially free of the l-threo isomer and of erythro methylphenidates.

In some embodiments of the present invention, the therapeutically effective amount is a bolus dose of D-threo methylphenidate. The dosage form may be suitable for oral administration in embodiments that may be preferred. The administration of the effective amount may be subcutaneous, intravenous, intramuscular, or interperitoneal. In some embodiments, the administration may also be via a pharmaceutical carrier selected from the group consisting of a sterile liquid or mixture of liquids, an alcohol, glycols, glycerol ketals, and ethers.

There are embodiments that may be preferred wherein the effective amount is 0.01% by weight of D-threo methylphenidate or salt thereof. The dosage form in some embodiments may have a viscosity increasing substance selected from the group consisting of sodium carboxymethylcellulose, sorbitol, dextran, and stabilizers. The bolus dosage form in other embodiments may be from about 0.01 mg/kg to about 1 mg/kg or from about 0.1 mg/kg to about 0.5 mg/kg of patient body weight. The bolus dosage form may also, understandably, comprise a pharmaceutically acceptable carrier. It will also be appreciated that pulsatile dosage forms are suitable for use in the present invention.

### DETAILED DESCRIPTION OF PREFERRED EMBODIMENTS

The present invention provides, in one aspect, methods for treating a class of patients suffering from both (a) Tourette's Syndrome and/or tics and (b) another disorder responsive to the administration of D-threo methylphenidate or a salt thereof, such as, for example, D-threo methylphenidate hydrochloride. The methods involve identifying a patient suffering from a disease or disorder, such as attention deficit disorder or attention deficit hyperactivity disorder, and having a family history or diagnosis of tics or Tourette's Syndrome and administering to said patient a therapeutically effective amount of D-threo methylphenidate substantially free of the l-threo isomer and of erythro methylphenidates. The D-threo methylphenidate may be administered in single, bolus dosages, with one dose being administered in each twenty-four hour period. The drug may also be administered by pulsatile dosage forms or dosage forms that yield two doses of drug.

Disorders responsive to the administration of D-threo methylphenidate or a salt thereof, such as, for example, D-threo methylphenidate hydrochloride, may be those described in U.S. Pat. No. 6,486,177, assigned to the assignees of the present application and incorporated herein by reference in its entirety. They may comprise fatigue, neurobehavioral slowing and cognitive side effects arising from cancer, or from a treatment therefor, such as chemotherapy, radiation therapy, administration of medication to control pain, and neurobehavioral slowing arising from the administration of a treatment for an oncological condition. Other disorders may comprise the symptoms of menopause, depression caused by cognitive dysfunction (a “cognitive side effect”) and fatigue associated with cancer, and treatments therefor. The treatment of an oncological condition may be considered to be the administration of pain management and biological therapies, comprising pain relief medication, chemotherapy, radiation therapy, and surgery. In some particularly preferred embodiments, the treatment for the oncological condition is chemotherapy or the administration of pain relief medication. In further embodiments of the disclosed methods, the pain relief medication is one or more opioid analgesics, nerve blocks, or other psychotropic agents. Other disorders responsive to the administration of D-threo methylphenidate or a salt thereof, such as, for example, D-threo methylphenidate hydrochloride, may be certain types of cognitive decline associated with patients suffering from Acquired Immunodeficiency Syndrome (AIDS) or AIDS-related conditions, including but not limited to AIDS-related dementia, as comprisd in U.S. Pat. No. 6,602,887, herein incorporated by reference in its entirety.

Disorders responsive to the administration of D-threo methylphenidate or a salt thereof, such as, for example, D-threo methylphenidate hydrochloride, may also comprise, for example, ADD and ADHD, as outlined in U.S. Pat. No. 6,528,530, herein incorporated by reference in its entirety.

Disorders responsive to the administration of D-threo methylphenidate or a salt thereof, such as, for example, D-threo methylphenidate hydrochloride, may also comprise, for example, those symptoms associated with menopause, comprising vasomotor instability, nervousness, excitability, fatigue, neurobehavioral slowing, apathy, mental depression and impairment of short term memory, as outlined in U.S. Pat. No. 6,486,177, herein incorporated by reference in its entirety.

According to one method of the present invention, bolus dosage forms are administered of D-threo methylphenidate substantially free of L-threo methylphenidate and of erythro methylphenidates. “Substantially free,” as used herein, refers to the presence of one optical isomer of a compound to the near or total exclusion of any other optical isomer of a compound. For example, in the context of the present invention, D-threo methylphenidate is “substantially free” of other optical isomers of methylphenidate within a dosage form if the amount of D-threo methylphenidate within the dosage form represents at least about 95%, at least about 96%, at least about 97%, at least about 98%, or at least about 99% of the total amount of methylphenidate in the dosage form. The D-threo form can be isolated by methods known to those skilled in the art.

“Chronic,” as used herein, refers to continuous, regular, long-term therapeutic administration, i.e. periodic administration without substantial interruption, such as, for example, daily, for a time period of at least several weeks or months to several years, for the purpose of treating a nervous disorder in a patient needing treatment.

“Bolus,” as used herein, refers to administration of a drug as a single event. The term “bolus” is intended to exclude dosage forms such as sustained release, pulsed release, and time release, and comprises any dosage form which can be used to deliver a single dose. According to the present invention, a bolus is preferably administered to a patient in need of treatment once daily, more preferably in the morning. The bolus dosages of the present invention may be administered in any conventional form known to those skilled in the art. Suitable methods for administration comprises oral dosage forms, injection, and infusion. Bolus dosage forms of methylphenidate drugs are taught by, for example, U.S. Pat. No. 6,602,887, incorporated herein by reference in its entirety.

The methods of the present invention may also be carried out by pulsatile dosage forms as described in U.S. Pat. No. 5,837,284 to Mehta et al., assigned to the assignee of the present application and incorporated herein by reference in its entirety. In such dosage forms, the release of the first dose preferably occurs substantially immediately; for example, release may occur within about 30 minutes following administration. Following a period of little or substantially no drug release, the second dose is released. Such a release profile may be referred to as “pulsatile.”

The release of the first dose may be within about a half hour following ingestion, preferably about 15 minutes, and more preferably within about 5 minutes following ingestion. The second, or delayed release, may comprise a period during which no more than about 10 percent of the drug in a particular dosage form is released, followed by a period of from about 0.5 hour to about 2.5 hours, preferably about 1.5 hours, more preferably about 1 hour, in which no less than about 70 percent, preferably no less than about 80 percent, and more preferably no less than about 90 percent, of the drug is released. If desired, a third release may follow in some embodiments where a suitable dosage form is used. Thus, dosage forms providing 3 or more doses are may be used in the present methods.

The doses delivered in the pulsatile forms may be varied in a number of ways. For example, the first dose can provide from about 30 percent to about 70 percent of a patient's daily prescribed intake of the drug and the second dose provides from about 70 percent to about 30 percent. If two approximately equal doses are desired, the initial dose preferably provides from about 40 percent to about 60 percent and the second dose preferably provides from about 60 percent to about 40 percent of a patient's prescribed daily intake of the drug. If desired, the first dose and the second dose can each provide about 50 percent of a patient's prescribed daily intake of drug. However, as will be apparent to one skilled in the art, the effect of drug metabolism in the body may require adjustment of the relative amounts of each dose, so that, for example, the second dose may have to be adjusted to provide more of the drug than the first dose to compensate for any competition between drug release and drug metabolism.

The delayed dosage forms may be achieved using methods known in the art. They may be provided in part by the use of certain copolymers referred to as “ammonio methacrylate copolymers.” Ammonio methacrylate copolymers comprise acrylic and/or methacrylic ester groups together with quaternary ammonium groups. The copolymers may be incorporated into a formulation which is used to coat particles containing a medication.

The acrylic and/or methacrylic ester groups in the copolymers used in the methods of the present invention may be referred to as “acrylic groups.” The acrylic groups are preferably derived from monomers selected from C₁-C₆ alkyl esters of acrylic acid and C₁-C₆ alkyl esters of methacrylic acid. Preferred may be C₁-C₄ alkyl esters of acrylic acid and methacrylic acid. Suitable monomers comprise, for example, methyl acrylate, ethyl acrylate, methyl methacrylate, and ethyl methacrylate. Ethyl acrylate and methyl methacrylate are preferred, and copolymers containing ethyl acrylate and methyl methacrylate are highly preferred. Also preferably, the copolymers have a molecular weight of about 150,000.

In embodiments utilizing the bolus dosage forms, the compounds described herein may be taken up in pharmaceutically acceptable carriers, such as, for example, solutions, suspensions, tablets, capsules, ointments, elixirs and injectable compositions. Pharmaceutical preparations generally can contain from about 1% to about 90% by weight of active ingredient. Preparations which are in single dose form, “unit dosage form,” preferably contain from about 20% to about 90% active ingredient. As used herein, the term “active ingredient” refers to compounds described herein, salts thereof, and mixtures of compounds described herein with other pharmaceutically active compounds. Dosage unit forms such as, for example, tablets or capsules, typically contain from about 0.001 g to about 1.0 g of active ingredient. Pharmaceutical preparations may be administered orally, parenterally, or topically.

Pharmaceutical preparations containing compounds described herein may be prepared by methods known to those skilled in the art, such as, for example, conventional mixing, granulating, dissolving, or lyophilizing. Oral dosage forms comprise capsules, pills, tablets, troches, lozenges, melts, powders, solutions, suspensions and emulsions. The oral dosage forms provided by the invention can be in the form of tablets, caplets, and the like and can be of any shape suitable for oral administration of a drug, such as spheroidal, cube-shaped, oval, bean shaped, or ellipsoidal. For oral dosage forms, for example, the compounds may be combined with one or more solid pharmaceutically acceptable carriers, optionally granulating the resulting mixture. One or more pharmaceutically acceptable adjuvants may optionally be included, such as, for example, flow-regulating agents and lubricants. Suitable carriers comprise, for example, fillers such as sugars, cellulose preparations, calcium phosphates; and binders such as methylcellulose, hydroxymethylcellulose, and starches, such as, for example, maize starch, potato starch, rice starch, and wheat starch. The dosage form may be in the form of granules, which may be irregularly shaped. The dosage form can comprise a capsule containing particles. Examples of orally administrable pharmaceutical preparations are dry-filled capsules consisting of gelatin, and soft sealed capsules consisting of gelatin and a plasticizer such as glycerol or sorbitol. The dry-filled capsules may contain the active ingredient in the form of a granulate, for example in admixture with fillers, binders, glidants, and stabilizers. In soft capsules, the active ingredient is preferably dissolved or suspended in a suitable liquid adjuvant, such as, for example, a fatty oil, paraffin oil, or liquid polyethylene glycol, optionally in the presence of stabilizers. Other oral administrable forms comprise syrups containing active ingredient, for example, in suspended form at a concentration of from about 0.01% to 20%, or in a similar concentration that provides a suitable single dose when administered, for example, in measures of from about 2 to about 5 milliliters. Suitable excipients for use in oral liquid dosage forms comprise diluents such as water and alcohols, for example ethanol, benzyl alcohol and polyethylene alcohols, either with or without the addition of a pharmaceutically acceptable surfactant, suspending agent, or emulsifying agent. Also suitable are powdered or liquid concentrates for combining with liquids such as milk. Such concentrates may also be packed in single dose quantities.

The compounds described herein may be administered parenterally, that is, subcutaneously, intravenously, intramuscularly, or interperitoneally, as injectable dosages of the compound in a physiologically acceptable diluent with a pharmaceutical carrier. Solutions for parenteral administration may be in the form of infusion solutions. A pharmaceutical carrier may be, for example, a sterile liquid or mixture of liquids such as water, saline, aqueous dextrose and related sugar solutions, an alcohol such as ethanol, glycols such as propylene glycol or polyethylene glycol, glycerol ketals such as 2,2-dimethyl-1,3-dioxolane-4-methanol, ethers such as poly(ethyleneglycol)400, oils, fatty acids, fatty acid esters or glycerides, with or without the addition of a pharmaceutically acceptable surfactant such as a soap or detergent, suspending agent such as pectin, carbomers, methylcellulose, hydroxypropylmethylcellulose, or carboxymethylcellulose, or emulsifying agent or other pharmaceutically acceptable adjuvants. Examples of oils which may be used in parenteral formulations comprise petroleum, animal, vegetable, or synthetic oils such as, for example, peanut oil, soybean oil, sesame oil, cottonseed oil, corn oil, olive oil, petrolatum, and mineral oil. Suitable fatty acids comprise, for example, oleic acid, stearic acid, and isostearic acid. Suitable fatty acid esters comprise ethyl oleate and isopropyl myristate. Suitable soaps comprise alkaline metal, ammonium and triethanolamine salts of fatty acids. Suitable detergents comprise cationic detergents such as dimethyl dialkyl ammonium halides and alkyl pyridinium halides; anionic detergents such as alkyl, aryl and olefin sulfonates, monoglyceride sulfates and sulfosuccinates; nonionic detergents such as fatty amine oxides, fatty acid alkanolamides and polyoxyethylenepropylene copolymers; and amphoteric detergents such as alkyl-(-aminopropionates and 2-alkylimidazoline quaternary ammonium salts; as well as mixtures of detergents. Parenteral preparations will typically contain at least about 0.01% by weight of active ingredient in solution. Preservatives and buffers may also be used advantageously. Injection suspensions may comprise viscosity-increasing substances such as, for example, sodium carboxymethylcellulose, sorbitol or dextran, and may also comprise stabilizers. In order to minimize irritation at the site of injection, injectable compositions may contain a non-ionic surfactant having a hydrophile-lipophile balance (HLB) of from about 12 to about 17. The quantity of surfactant in such formulations ranges from about 5% to about 15% by weight. The surfactant may be a single component having the above HLB or a mixture of two or more components having the desired HLB. Particular examples of useful surfactants comprise polyethylene sorbitan fatty acid esters, such as, for example, sorbitan monooleate.

The preferred quantity of D-threo methylphenidate to be used in a dosage for treating a particular patient can be readily determined by one skilled in the art. Factors determining the appropriate dosage include, for example, the weight and age of the patient, the type and extent of the disorder being treated, and other conditions of the patient comprising other disorders and other medications, if any, that the patient is taking. Generally, the dosage of D-threo methylphenidate will be from about 0.01 mg/kg of patient body weight to about 1 mg/kg of patient body weight. Appropriate quantities can be determined by one skilled in the art. For example, a relatively small child may generally require a dose of from about 0.03 to about 0.3 mg/kg, while a larger child or an adult may require a dose of from about 0.1 mg/kg to about 0.4 or 0.5 mg/kg.

What is claimed:

1. A method for treating a disease or disorder responsive to the administration of D-threo methylphenidate or a salt thereof, said method comprising the steps of:
identifying a patient suffering from said disease or disorder and suffering from Tourette's Syndrome or tics or having a family history of Tourette's Syndrome or tics; and
administering to said patient a dosage form comprising a therapeutically effective amount of D-threo-methylphenidate or a salt thereof, said D-threo methlyphenidate substantially free of both the l-threo isomer and salts thereof and the erythro methylphenidates and salts thereof.

2. The method of claim 1 wherein said therapeutically effective amount is a bolus dose.

3. The method of claim 1 wherein said dosage form is suitable for oral administration.

4. The method of claim 1 wherein said administration is subcutaneous, intravenous, intramuscular, or interperitoneal.

5. The method of claim 1 wherein said administration is via a pharmaceutical carrier selected from the group consisting of a sterile liquid or mixture of liquids, an alcohol, glycols, glycerol ketals, and ethers.

6. The method of claim 5 wherein said sterile liquid or mixture of liquids is water, saline, aqueous dextrose, or related sugar solutions.

7. The method of claim 5 wherein said alcohol is ethanol.

8. The method of claim 5 wherein said glycols are propylene glycol or polyethylene glycol.

9. The method of claim 5 wherein said glycerol ketal is 2,2-dimethyl-1,3-dioxolane-4-methanol.

10. The method of claim 5 wherein said ether is poly(ethyleneglycol)400, oils, fatty acids, fatty acid esters, or glycerides.

11. The method of claim 10 further comprising at least one pharmaceutically acceptable surfactant, a suspending agent, an emulsifying agent, or other pharmaceutically acceptable adjuvants.

12. The method of claim 11 wherein said surfactant is a soap, detergent, or mixture of detergents.

13. The method of 11 wherein said suspending agent is pectin, carbomers, methylcellulose, hydroxypropylmethylcellulose, or carboxymethylcellulose.

14. The method of claim 10 wherein said oils are selected from the group consisting of petroleum, animal, vegetable, or synthetic oils.

15. The dosage method of 14 wherein said oils are peanut oil, soybean oil, sesame oil, cottonseed oil, corn oil, olive oil, petrolatum, or mineral oil.

16. The method of claim 10 wherein said fatty acids are selected from the group consisting of oleic acid, stearic acid, and isostearic acid.

17. The method of claim 10 wherein said fatty acid esters are selected from the group consisting of ethyl oleate and isopropyl myristate.

18. The method of claim 12 wherein said soap is alkaline metal, ammonium and triethanolamine salts of fatty acids.

19. The method of claim 12 wherein said detergent is selected from the group consisting of cationic detergents, anionic detergents, nonionic detergents, and amphoteric detergents.

20. The method of claim 19 wherein said detergent is dimethyl dialkyl ammonium halides, alkyl pyridinium halides, alkyl, aryl and olefin sulfonates, monoglyceride sulfates, sulfasucciantes, fatty amine oxides, fatty acid alkanolamides, polyoxyethylenepropylene copolymers, alkyl-aminopropoionates, or 2-alkylimidazoline quaternary ammonium salts.

21. The method of claim 1 wherein said therapeutically effective amount is 0.01% by weight of D-threo methylphenidate or salt thereof.

22. The method of claim 1 further comprising administering a viscosity increasing substance selected from the group consisting of sodium carboxymethylcellulose, sorbitol, dextran, and stabilizers.

23. The method of claim 11 wherein said surfactant is about 5% to about 15% by weight of the dosage form.

24. The method of claim 11 wherein said surfactant is selected from the group consisting of polyethene sorbitan fatty acid esters.

25. The method of claim 24 wherein the surfactant is sorbitan monooleate.

26. The method of claim 1 wherein said disorder is attention deficit-hyperactivity disorder, symptoms associated with menopause, or one or more of a decrease in cognitive function, fatigue, and neurobehavioral slowing that is unrelated to the administration of analgesics, but may be related to an underlying cancer, the treatment of the cancer, or both.

27. The method of claim 1 wherein said disorder is fatigue, neurobehavioral slowing and cognitive side effects arising from cancer, or from a treatment therefor, such as chemotherapy, radiation therapy, administration of medication to control pain, or neurobehavioral slowing arising from the administration of a treatment for an oncological condition.

28. The method of claim 1 wherein said administration is by pulsatile dosage forms.

29. The method of claim 1 wherein said dosage forms give two doses of drug.
